(19) United States
(12) Patent Application Publication
(10) Pub. No.: US 2011/0236307 A1
(43) Pub. Date: Sep. 29, 2011

(54) IN VIVO IMAGING METHOD

(76) Inventor: Paul Alexander Jones, Amersham (GB)

(21) Appl. No.: 13/132,109

(22) PCT Filed: Dec. 1, 2009
(86) PCT No.: PCT/EP09/66120

§ 371 Date: Jun. 1, 2011

Related U.S. Application Data
(60) Provisional application No. 61/119,060, filed on Dec. 2, 2008.

(30) Foreign Application Priority Data
Dec. 2, 2008 (GB) 0821994.1

(51) Int. Cl. A61K 51/10; A61K 49/00; A61K 51/04; A61K 49/10; A61K 49/16

(52) U.S. Cl. 424/1.49; 424/9.1; 424/1.65; 424/1.85; 424/9.3

(57) ABSTRACT

The present invention provides an in vivo imaging method that facilitates the diagnosis of Parkinson's disease (PD) at an early stage. Early diagnosis is particularly advantageous as neuroprotective treatment can be applied to healthy neural cells to delay or even prevent the onset of debilitating clinical symptoms.

### TECHNICAL FIELD OF THE INVENTION

The present invention relates to in vivo imaging and in particular to an in vivo imaging method to facilitate the early diagnosis of Parkinson's disease.

### DESCRIPTION OF RELATED ART

Braak et at (2004 Cell Tissue Res., 318: 121-34) have defined six stages in the neuropathophysiology of Parkinson's disease (PD), each successive stage defined by the progressive development of Lewy bodies (LB) and Lewy neurites (LN). These LB and LN consist mostly of aggregations of the protein α-synuclein (Spillantini et al 1997 Nature; 388: 839-40), which is found in healthy nerve cells as an unfolded membrane-bound protein. Under as yet undefined conditions, α-synuclein detatches from the membrane and takes on a β-sheet conformation which permits aggregation and consequent formation of LB and LN. In PD, the earliest lesions appear at the olfactory bulb, anterior olfactory nucleus, and the dorsal motor nucleus of the vagus nerve (Braak 2004 Cell Tissue Res.; 318: 121-34). It has been hypothesised that this process might originate outside the central nervous system (CNS) triggered by an unknown pathogen that passes the mucosal barrier of the gastrointestinal tract (GIT) and enters the CNS through the vagus nerve via enteric neurons (Braak et al J. Neural Transm. 2003, 110: 517-36).

A fairly clear diagnosis of PD can in most cases be obtained using patient history and clinical examination. As discussed by Samii et al, (Lancet 2004; 363(9423): 1783-93) one of the criteria used for diagnosis is definitive response to anti-Parkinson's drugs, typically a dopamine agonist or levodopa. So, for example, a trial of levodopa can help to distinguish PD from normal ageing, essential tremor, corticobasal degeneration, multiple system atrophy (MSA), and dementia with Lewy bodies (DLB). However, exposure of a subject to an inappropriate treatment is not ideal. Apart from the unnecessary exposure to a range of potential side effects, in some cases the disease can be worsened. For example, for a subject whose is suffering from DLB, inappropriate treatment with anti-Parkinson drugs can worsen the psychiatric symptoms.

In vivo imaging of the CNS to assist in the diagnosis of PD is known in the art (see Rachakonda et al 2004 Cell Res., 14(15): 349-60). For example, 6-[¹⁸F]-fluoro-L-dopa is used as a PET tracer to evaluate the function of dopaminergic neurons. The SPECT tracer [¹²³I]-2-[β]-carbomethoxy-3-[β]-(4-iodophenyl)-tropane is used to evaluate the function of the monoamine vesicular transporter. WO 2004/075882 discloses an in vivo imaging method to diagnose the presence of abnatmally folded or aggregated protein and/or amyloid fibril or amyloid in a subject where the method comprises administration of a radiolabelled inositol derivative. In WO 2004/075882 it is taught that the in vivo imaging method can be applied for the diagnosis of PD; but there is no mention in WO 2004/075882 of in vivo imaging of PD by targeting abnormally folded or aggregated protein outside the CNS. There is also no specific disclosure in WO 2004/075882 that the abnormally folded or aggregated protein is α-synuclein.

In vivo imaging agents have been reported that particularly target α-synuclein deposits present in the central nervous system (CNS) of subjects suffering from PD. WO 2004/100998 discloses agents that bind amyloid-β labelled with an in vivo imaging agent and teaches that these compounds can also be used to target α-synuclein deposits in the CNS to help diagnose PD. WO 2005/013889 provides a method for in vivo imaging of LB to diagnose a LB disease, said method comprising administration to a patient of an antibody that specifically binds to α-synuclein in LB. WO 2005/013889 describes LB disease in terms of the presence of LB in the CNS and makes no particular mention of LB outside the CNS.

Although the above-described in vivo imaging techniques may overcome the problem of inaccurate differential diagnosis and inappropriate application of PD treatment, they all target the disease process at a stage when LB and LN are present in the CNS. At this stage, clinical symptoms are evident, and about 80% of striatal dopamine neurons and 50% of nigral neurons are lost (Samii et al 2004 The Lancet; 363(9423): 1783-1793). As the neurons of the CNS cannot regenerate on their own after cell death, neuroprotective treatment will only benefit neurons still alive at the time of diagnosis. It would be advantageous for patients to get treatment to curb disease progression as early as possible. There is therefore a need for a method to identify PD before significant loss of neurons.

### SUMMARY OF THE INVENTION

The present invention provides an in vivo imaging agent for use in a method for the diagnosis of Parkinson's disease (PD) at an early stage. Early diagnosis is particularly advantageous as neuroprotective treatment can be applied to healthy neural cells to delay or even prevent the onset of debilitating clinical symptoms. A further advantage of the present invention over the prior art is that the in vivo imaging agent does not have to get into the CNS. Therefore it is not necessary to consider whether the in vivo imaging agent will penetrate the blood brain barrier, or to consider the relatively invasive route of direct administration of an in vivo imaging agent to the brain.

### DETAILED DESCRIPTION OF THE INVENTION

### Method of Imaging

In one aspect, the present invention provides an in vivo imaging agent for use in a method to determine the presence of, or susceptibility to, Parkinson's disease (PD), wherein said in vivo imaging agent comprises an α-synuclein binder labelled with an in vivo imaging moiety, and wherein said in vivo imaging agent binds to α-synuclein with a binding affinity of 0.1 nM-50 μM, said method comprising:
(i) administering to a subject a detectable quantity of said in vivo imaging agent;
(ii) allowing said administered in vivo imaging agent of step (i) to bind to α-synuclein deposits in the autonomic nervous system (ANS) of said subject;
(iii) detecting signals emitted by said bound in vivo imaging agent of step (ii) using an in vivo imaging method;
(iv) generating an image representative of the location and/or amount of said signals; and,
(v) using the image generated in step (iv) to determine of the presence of, or susceptibility to, PD.
   The term “α-synuclein deposits” refers to insoluble proteinaceous inclusions comprising the protein α-synuclein. Lewy bodies (LB) and Lewy neurites (LN) are well-known insoluble proteinaceous inclusions wherein α-synuclein is the main component, and in PD have been reported to be present in the central nervous system (CNS) as well as in the ANS. However, PD is conventionally considered as a disease of the CNS, and known in vivo imaging methods for the detection of PD target α-synuclein deposits present in the CNS.

The “central nervous system” (CNS) is that part of the nervous system in vertebrates consisting of the brain and the spinal cord. In the CNS, endothelial cells are packed together more tightly than in the rest of the body by means of “tight junctions”, which are multifunctional complexes that form a seal between adjacent epithelial cells, preventing the passage of most dissolved molecules from one side of the epithelial sheet to the other. This forms the blood-brain barrier (BBB), which blocks the movement of all molecules except those that cross cell membranes by means of lipid solubility (such as oxygen, carbon dioxide, ethanol, and steroid hormones) and those that are allowed in by specific transport systems (such as sugars and some amino acids). Substances with a molecular weight higher than 500 Da (such as antibodies) generally cannot cross the BBB by passive diffusion, while smaller molecules often can. In order for an in vivo imaging agent to come into contact with a target in the CNS, its chemical structure has to be tailored for passage across the BBB, or alternatively the in vivo imaging agent has to be administered directly into the CNS using relatively invasive procedures.

The peripheral nervous system (PNS) resides or extends outside the CNS. Unlike the CNS, the PNS is not protected by the BBB. The peripheral nervous system is divided into the somatic nervous system and the autonomic nervous system. The “autonomic nervous system” (ANS) (also known as the visceral nervous system) is the part of the PNS that acts as a control system, maintaining homeostasis in the body. These activities are generally performed without conscious control or sensation. Whereas most of its actions are involuntary, some, such as breathing, work in tandem with the conscious mind. Its main components are its sensory system, motor system (comprised of the parasympathetic nervous system and sympathetic nervous system), and the enteric nervous system (ENS; controls the gastrointestinal system).

The method of the invention begins by administering a detectable quantity of an in vivo imaging agent to a subject. Since the ultimate purpose of the method is the provision of a diagnostically-useful image, administration to the subject of said in vivo imaging agent can be understood to be a preliminary step necessary for facilitating generation of said image. In an alternative embodiment the method of the invention can be said to begin by providing a subject to whom a detectable quantity of an in vivo imaging agent has been administered. “Administering” the in vivo imaging agent means introducing the in vivo imaging agent into the subject's body, and is preferably carried out parenterally, most preferably intravenously. The intravenous route represents the most efficient way to deliver the in vivo imaging agent throughout the body of the subject.

The “subject” of the invention is preferably a mammal, most preferably an intact mammalian body in vivo. In an especially preferred embodiment, the subject of the invention is a human.

The term “in vivo imaging agent” broadly refers to a compound which can be detected following its administration to the mammalian body in vivo. The in vivo imaging agent of the present invention comprises an α-synuclein binder labelled with an in vivo imaging moiety. The term “labelled with an in vivo imaging moiety” means either (i) that a particular atom of the α-synuclein binder is an isotopic version suitable for in vivo detection, or (ii) that a group comprising said in vivo imaging moiety is conjugated to said α-synuclein binder. Examples of both are described in more detail below. The in vivo imaging agent has binding affinity for α-synuclein in the range 0.1 nM-50 μM, preferably 0.1 nM-1 μM and most preferably 0.1-100 nM. Masuda et al (2006 Biochemistry; 45: 6085-94) describe an assay for testing the ability of compounds to bind to α-synuclein in vitro. In the assay, a test compound is incubated with a solution of α-synuclein at 37° C. for 72 hours, followed by addition of the detergent sarkosyl (sodium lauroyl sarcosinate) to facilitate determination of the relative proportions of soluble and insoluble α-synuclein. IC₅₀ values for the test compounds can be calculated by quantifying the amount of sarkosyl-insoluble α-synuclein. This assay can therefore be used to test the suitability of a particular in vivo imaging agent for the present invention. There are a variety of compounds that are known to have binding affinity for α-synuclein, and which are therefore suitable as a basis for obtaining in vivo imaging agents suitable for the present invention. Matsuda et al (supra) disclose a range of different compound classes that bind to α-synuclein. In addition, antibodies specific for α-synuclein are known and commercially available from a number of sources. Non-limiting examples of some preferred α-synuclein binders and corresponding in vivo imaging agents are described in more detail below.

An “in vivo imaging moiety” may be detected either externally to the human body, or via use of detectors designed for use in vivo, such as intravascular radiation or optical detectors such as endoscopes, or radiation detectors designed for intra-operative use.

Following the administering step and preceding the detection step, the in vivo imaging agent is allowed to bind to α-synuclein deposits in the ANS of said subject. For example, when the subject is an intact mammal, the in vivo imaging agent will dynamically move through the mammal's body, coming into contact with various tissues therein. Once the in vivo imaging agent comes into contact α-synuclein, a specific interaction takes place such that clearance of the in vivo imaging agent from tissue with α-synuclein takes longer than from tissue without, or with less α-synuclein. A certain point in time will be reached when detection of in vivo imaging agent specifically bound to α-synuclein is enabled as a result of the ratio between in vivo imaging agent bound to tissue with α-synuclein versus that bound in tissue without, or with less α-synuclein. An ideal such ratio is at least 2:1. Preferably, said α-synuclein deposits are present in the ENS, i.e. the myenteric (Auerbach's) and submucosal (Meissner's) plexuses of the gut.

The “detection” step of the method of the invention involves the detection of signals either externally to the human body or via use of detectors designed for use in vivo, such as intravascular radiation or optical detectors such as endoscopes (e.g. suitable for detection of signals in the gut), or radiation detectors designed for intra-operative use. This detection step can also be understood as the acquisition of signal data.

The “in vivo imaging method” selected for detection of signals emitted by said in vivo imaging moiety depends on the nature of the signals. Therefore, where the signals come from a paramagnetic metal ion, magnetic resonance imaging (MRI) is used, where the signals are gamma rays, single photon emission tomography (SPECT) is used, where the signals are positrons, positron emission tomography (PET) is used, and where the signals are optically active, optical imaging is used. All are suitable for use in the method of the present invention, with PET and SPECT are preferred, as they are least likely to suffer from background and therefore are the most diagnostically useful.

The “generation” step of the method of the invention is carried out by a computer which applies a reconstruction algorithm to the acquired signal data to yield a dataset. This dataset is then manipulated to generate images showing areas of interest within the subject.

### Preferred In Vivo Imaging Moieties

The in vivo imaging moiety is preferably chosen from:
(i) a radioactive metal ion;
(ii) a paramagnetic metal ion;
(iii) a gamma-emitting radioactive halogen;
(iv) a positron-emitting radioactive non-metal;
(v) a reporter suitable for in vivo optical imaging.
   In vivo imaging agents may be conveniently prepared by reaction of a precursor compound with a suitable source of the in vivo imaging moiety. A “precursor compound” comprises a derivative of the in vivo imaging agent, designed so that chemical reaction with a convenient chemical form of the in vivo imaging moiety occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. The precursor compound may optionally comprise a protecting group for certain functional groups of the precursor compound.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection, the desired in vivo imaging agent is obtained. Protecting groups are well-known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde (i.e. 1-(4,4-dimethyl-2,6-dioxocyclohexylidene)ethyl) or Npys (i.e. 3-nitro-2-pyridine sulfenyl); and for carboxyl groups: methyl ester, tert-butyl ester or benzyl ester.

For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of protecting groups is described in ‘Protective Groups in Organic Synthesis’, Theorodora W Greene and Peter G. M. Wuts, (Third Edition, John Wiley & Sons, 1999).

When the in vivo imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the in vivo imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety comprises a metal ion, it is preferably present as a metal complex of the metal ion with a synthetic ligand. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include other excipients in the preparation in vitro (e.g. radioprotectants or antimicrobial preservatives used in the preparation), or endogenous compounds in vivo (e.g. glutathione, transferrin or plasma proteins). The term “synthetic” has its conventional meaning, i.e. man-made as opposed to being isolated from natural sources e.g. from the mammalian body. Such compounds have the advantage that their manufacture and impurity profile can be fully controlled.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as MIBI (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

When the metal ion is technetium, suitable chelating agents which form metal complexes resistant to transchelation include, but are not limited to:

(i) diaminedioximes;
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam dioxocyclam; and,
(v) N₂O₂ ligands having a diaminediphenol donor set.

Examples of chelates that are particularly suitable for complexing ^{99m}Tc are described in WO 2003/006070 and WO 2006/008496.

When the in vivo imaging moiety is a gamma-emitting radioactive halogen, the radiohalogen is suitably chosen from ¹²³I, ¹³¹I or ⁷⁷Br. ¹²⁵I is specifically excluded as it is not suitable for use as an imaging moiety for in vivo diagnostic imaging.

Where a compound is labelled with a gamma-emitting radioactive halogen, suitable precursor compounds are those which comprise a derivative which either undergoes electrophilic or nucleophilic halogenation or undergoes condensation with a labelled aldehyde or ketone. Examples of the first category are:
(a) organometallic derivatives such as a trialkylstannane (eg. trimethylstannyl or tributylstannyl), or a trialkylsilane (eg. trimethylsilyl) or an organoboron compound (eg. boronate esters or organotrifluoroborates);
(b) a non-radioactive alkyl bromide for halogen exchange or alkyl tosylate, mesylate or triflate for nucleophilic halogenation;
(c) aromatic rings activated towards electrophilic halogenation (e.g. phenols, phenylamines) and aromatic rings activated towards nucleophilic halogenation (e.g. aryl iodonium salt aryl diazonium, aryl trialkylammonium salts or nitroaryl derivatives).
   The precursor compound for radiohalogenation preferably comprises: a non-radioactive halogen atom such as an aryl iodide or bromide (to permit radioiodine exchange); an activated aryl ring (e.g. a phenol or phenylamine); an organometallic substituent (e.g. trialkyltin, trialkylsilyl or organoboron compound); or an organic substituent such as triazenes or a good leaving group for nucleophilic substitution such as an iodonium salt. Preferably for radiohalogenation, the precursor compound comprises an activated aryl ring or an organometallic substituent, said organometallic substituent most preferably being trialkyltin.

A preferred gamma-emitting radioactive halogen is radioiodine, and in particular ¹²³I. Precursor compounds and methods of introducing radioiodine into organic molecules are described by Bolton (J. Lab. Comp. Radiopharm., 2002, 45: 485-528). Suitable boronate ester organoboron compounds and their preparation are described by Kabalaka et al (Nucl. Med. Biol., 2003; 29: 841-843 and 30: 369-373). Suitable organotrifluoroborates and their preparation are described by Kabalaka et al (Nucl. Med. Biol., 2004; 31: 935-938).

Examples of aryl groups to which radioactive iodine can be attached are given below:

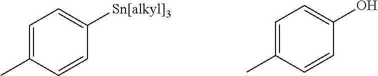

wherein alkyl in this case is preferably methyl or butyl. These groups contain substituents which permit facile radioiodine substitution onto the aromatic ring. Alternative substituents containing radioactive iodine can be synthesised by direct iodination via radioiodine exchange, e.g.:

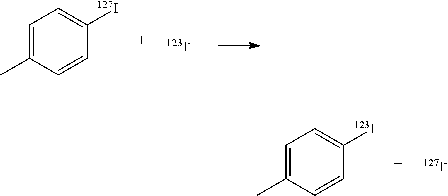

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

The source of the radioiodine is chosen from iodide ion or the iodonium ion (r). Most preferably, the chemical form is iodide ion, which is typically converted to an electrophilic species by an oxidant during radiosynthesis.

When the in vivo imaging moiety is a positron-emitting radioactive non-metal, suitable such positron emitters include: ¹¹ C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F. Techniques for introduction of these in vivo imaging moieties are well-known to those of skill in the art of positron emission tomography (PET) imaging. Some of these techniques are now described.

Where a compound is labelled with ¹¹C, one approach to labelling is to react a precursor compound which is the desmethylated version of a methylated compound with [¹¹C]methyl iodide. It is also possible to incorporate ¹¹C by reacting Grignard reagent of the particular hydrocarbon chain of the desired labelled compound with [¹¹C]CO₂. ¹¹C could also be introduced as a methyl group on an aromatic ring, in which case the precursor compound would include a trialkyltin group or a B(OH)₂ group.

As the half-life of ¹¹C is only 20.4 minutes, it is important that the intermediate ¹¹C moieties have high specific activity and consequently are produced using a reaction process which is as rapid as possible.

A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

To label a compound with a radioactive isotope of fluorine the radiofluorine atom may form part of a fluoroalkyl or fluoroalkoxy group, since alkyl fluorides are resistant to in vivo metabolism. Fluoroalkylation may be carried out by reaction of a precursor compound containing a reactive group such as phenol, thiol and amide with a fluoroalkyl group.

Alternatively, the radiofluorine atom may be attached via a direct covalent bond to an aromatic ring such as a benzene ring. For such aryl systems, ¹⁸F-fluoride nucleophilic displacement from an aryl diazonium salt, aryl nitro compound or an aryl quaternary ammonium salt are suitable routes to aryl-¹⁸F derivatives.

Radiofluorination may be carried out via direct labelling using the reaction of ¹⁸F-fluoride with a suitable chemical group in the precursor compound having a good leaving group, such as an alkyl bromide, alkyl mesylate or alkyl tosylate.

As the half-life of ¹⁸F is only 109.8 minutes, it is important that the intermediate ¹⁸F moieties have high specific activity and, consequently, are produced using a reaction process which is as rapid as possible.

Further details of synthetic routes to ¹⁸F-labelled derivatives are described by Bolton, J. Lab. Comp. Radiopharm., 2002; 45: 485-528.

When the in vivo imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, naphthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 nm and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

In the present invention it is notable that some suitable α-synuclein binders are also reporters suitable for in vivo optical imaging. Where this is the case, the in vivo imaging agent is also the α-synuclein binder. Examples of such α-synuclein binders include derivatives of Thioflavin T and of Congo Red, which are described in more detail below. These compounds can alternatively be labelled with other in vivo imaging moieties if desired.

In a preferred embodiment, the in vivo imaging moiety of the present invention is a radioactive metal ion, a gamma-emitting radioactive halogen, or a positron-emitting radioactive non-metal. The suitable and preferred embodiments of each are as presented above. Particularly preferred in vivo imaging moieties of the present invention are ^{99m}Tc, ¹¹C, ¹⁸F and ¹²³I.

### Thioflavin T Derivatives

In a study of PD patients by Maetzler et al (NeuroImage 2008; 39(3): 1027-33) it was found that a compound within the scope of Formula Ia (described below), [¹¹C]PIB (¹¹C-6-OH-benzothiazole), had the potential to differentiate PD from Alzheimer's disease (AD). The in vivo binding pattern of [¹¹C]PIB in PD patients decreased from the brainstem to the cortical areas, correlating with the known sequence of protein deposition in PD pathophysiology (Braak et al 2004 Cell Tissue Res.; 318: 121-34). The in vitro binding of fluorescent PIB was also evaluated by Maetzler et al (supra), and it was observed to bind specifically to Lewy bodies in brainstem tissue of PD patients.

In addition, WO 2004/083195 discloses Thioflavin T derivatives labelled with a variety of in vivo imaging moieties for use in imaging β-amyloid plaques in the CNS to help in diagnosing Alzheimer's disease.

Volkova et al (Bioorg. Med. Chem. 2008; 16: 1452-9) report the specific histological detection of α-synuclein using mono- and trimethine cyanine dyes of Formula Ib. Derivatives of these dyes are therefore proposed by the present inventors to be useful in the present invention.

In one preferred embodiment, said α-synuclein binder is a compound of Formula I or Formula I(i):

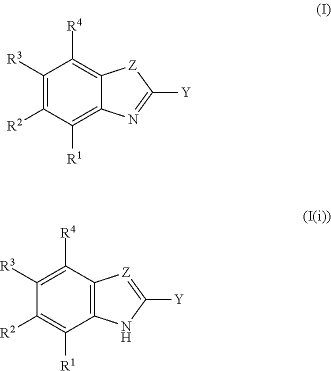

or a salt or solvate thereof, wherein:
R¹⁻⁴ are each independently hydrogen, or an R group selected from, C₁₋₆ alkyl, C₂₋₆ alkenyl, C₂₋₆ alkynyl, C₁₋₆ alkoxy, C₄₋₆ cycloalkyl, hydroxyl, C₁₋₆ hydroxyalkyl, C₂₋₆ hydroxyalkenyl, C₂₋₆ hydroxyalkynyl, thiol, C₁₋₆ thioalkyl, C₂₋₆ thioalkenyl, C₂₋₆ thioalkynyl, C₁₋₆ thioalkoxy, carboxyl, C₁₋₆ carboxyalkyl, halo, C₁₋₆ haloalkyl, C₂₋₆ haloalkenyl, C₂₋₆ haloalkynyl, C₁₋₆ haloalkoxy, amino, C₁₋₆ aminoalkyl, C₂₋₆ aminoalkenyl, C₂₋₆ aminoalkynyl, C₁₋₆ aminoalkoxy, cyano, C₁₋₆ cyanoalkyl, C₂₋₆ cyanoalkenyl, C₂₋₆ cyanoalkynyl, and C₁₋₆ cyanoalkoxy; nitro, C₁₋₆ nitroalkyl, C₂₋₆ nitroalkenyl, C₂₋₆-nitroalkynyl, C₁₋₆ nitroalkoxy, and —OCH₂OR′, wherein R′ is H or C₁₋₆ alkyl;
Y is a C₃₋₁₀ 5- to 10-membered aryl ring system having 0-3 heteroatoms selected from S, O and N, and 0-5 substituents each of which is an R group as defined for R¹⁻⁴;
in Formula I Z is S, O, or NR″ wherein R″ is hydrogen or C₁₋₃ alkyl; and,
in Formula I(i) Z is CR″ wherein R″ is as defined for NR″.

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those foamed with ethanol, water, saline, physiological buffer and glycol.

The term “alkyl” alone or in combination, means a straight-chain or branched-chain alkyl radical containing preferably from 1 to 6 carbon atoms, more preferably from 1 to 4 carbon atoms, most preferably 1 to 3 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl.

The term “alkenyl” denotes an unsaturated straight-chain or branched aliphatic hydrocarbon group containing one double bond. Examples groups such as vinyl (ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” denotes an unsaturated straight-chain or branched aliphatic hydrocarbon group containing one triple bond. Examples include groups such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Unless otherwise specified, the term “alkoxy”, alone or in combination, means an alkyl ether radical wherein the term alkyl is as defined above. Examples of suitable alkyl ether radicals include, but are not limited to, methoxy, ethoxy, n-propoxy, isopropoxy, n-butoxy, iso-butoxy, sec-butoxy, tert-butoxy.

Unless otherwise specified, the term “cycloalkyl”, alone or in combination, means a saturated or partially saturated monocyclic, bicyclic or tricyclic alkyl radical wherein each cyclic moiety contains preferably from 3 to 8 carbon atom ring members, more preferably from 3 to 7 carbon atom ring members, most preferably from 4 to 6 carbon atom ring members, and which may optionally be a benzo fused ring system which is optionally substituted as defined herein with respect to the definition of aryl. Examples of such cycloalkyl radicals include, but are not limited to, cyclopropyl, cyclobutyl, cyclopentyl, cyclohexyl, cycloheptyl, octahydronaphthyl, 2,3-dihydro-1H-indenyl, adamantyl.

The term “hydroxyl” refers to a —OH group. The terms “hydroxyalkyl”, “hydroxyalkenyl” and “hydroxyalkynyl”, as used herein, refer to at least one hydroxy group appended to the parent molecular moiety through an alkyl, alkenyl, alkynyl, or alkoxy, respectively.

The term “halo” means a substituent selected from fluorine, chlorine, bromine or iodine. The terms “haloalkyl”, “haloalkenyl”, “haloalkynyl”, “haloalkoxy” as used herein, refer to at least one halo group appended to the parent molecular moiety through an alkyl, alkenyl, alkynyl, or alkoxy, respectively. Preferred halo substituents are fluoro and iodo.

The term “thiol” means an —SH group. The terms “thioalkyl”, “thioalkenyl”, “thioalkynyl”, “thioalkoxy” as used herein, refer to at least one thiol group appended to the parent molecular moiety through an alkyl, alkenyl, alkynyl, or alkoxy, respectively.

The term “cyano” as used herein refers to a —CN group. The terms “cyanoalkyl”, “cyanoalkenyl”, “cyanoalkynyl”, “cyanoalkoxy” as used herein, refer to at least one cyano group appended to the parent molecular moiety through an alkyl, alkenyl, alkynyl, or alkoxy, respectively. Representative examples of cyanoalkyl include, but are not limited to, cyanomethyl, 2-cyanoethyl, and 3-cyanopropyl.

The term “nitro” means an —NO₂ group. The terms “nitroalkyl”, “nitroalkenyl”, “nitroalkynyl”, “nitroalkoxy” as used herein, refer to at least one nitro group appended to the parent molecular moiety through an alkyl, alkenyl, alkynyl, or alkoxy, respectively.

The term “amino” means the group —NR⁹R¹⁰, wherein R⁹ and R¹⁰ are independently hydrogen or an R group as defined above for Formula I. The terms “aminoalkyl”, “aminoalkenyl”, “aminoalkynyl”, “aminoalkoxy” as used herein, refer to at least one amino group appended to the parent molecular moiety through an alkyl, alkenyl, alkynyl, or alkoxy, respectively.

The term “carboxyl” means the group —COOH and the term “carboxyalkyl” refers to an alkyl group as defined herein wherein at least one carboxyl group is appended to the parent molecular moiety.

“Aryl” means aromatic rings or ring systems having 3 to 10 carbon atoms, and 5-10 members, in the ring system, e.g. phenyl or naphthyl. The term “heteroatom” refers to a N, S or O atom taking the place of a carbon in the ring system.

In a preferred embodiment, when said α-synuclein binder is a compound of Formula I, said in vivo imaging agent is a compound of Formula Ia:

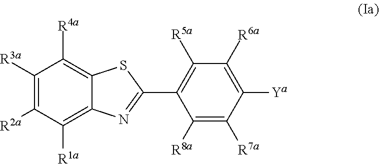

or a salt or solvate thereof, wherein:
each R^{1a}-R^{8a} is independently hydrogen or an R group as defined above for Formula I, or comprises an in vivo imaging moiety as defined herein; and,
Y^{a} is hydrogen, C₁₋₆ alkyl, halo, hydroxyl, C₁₋₆ hydroxyalkyl, thiol, C₁₋₆ thioalkyl, or Y^{a} is an amino group —NR⁹R¹⁰, wherein R⁹ and R¹⁰ are independently hydrogen or an R group as defined in claim 3, or Y^{a} is an in vivo imaging moiety as defined herein;
wherein at least one of R^{1a}-R^{8a} and Y^{a} comprises an in vivo imaging moiety as defined herein.

Preferably for Formula Ia:
each R^{1a-8a} is independently selected from hydrogen, nitro, cyano, C₁₋₆ alkyl, C₂₋₆ alkenyl, C₂₋₆ alkynyl, C₁₋₆ alkoxy, hydroxyl, C₁₋₆ hydroxyalkyl, halo, C₁₋₆ haloalkyl, C₁₋₆ haloalkoxy, C₁₋₆ haloalkenyl, carboxyl, C₁₋₆ carboxyalkyl, —OCH₂OR′ wherein R′ is hydrogen or C₁₋₃ alkyl; or, or each R^{1a-8a} independently comprises an in vivo imaging moiety as defined herein;
Y^{a} is —NR⁹R¹⁰ or comprises an in vivo imaging moiety as defined herein; and,
wherein at least one of R^{1a-8a} and Y^{a} comprises an in vivo imaging moiety as defined herein.

Most preferably for Formula Ia:
R^{1a}, R^{2a}, R^{4a}, R^{7a}, and R^{8a} are all hydrogen;
R^{3a} is selected from hydrogen, hydroxyl, C₁₋₄ alkyl, C₂₋₄ alkenyl, C₂₋₄ alkynyl, C₁₋₄ alkoxy, halo, C₁₋₄ haloalkyl, C₁₋₄ haloalkenyl, carboxyl, C₁₋₄ carboxyalkyl, and —OCH₂OR′, wherein R′ is as defined above for Formula I and I(i); or, R^{3a} comprises an in vivo imaging moiety as defined herein; and,
R^{5a} and R^{6a} are each independently hydrogen, C₁₋₆ alkyl, C₁₋₆ alkoxy, nitro, amino, C₁₋₆ aminoalkyl, halo or C₁₋₆ haloalkyl; or, R^{5a} and R^{6a} each independently comprise an in vivo imaging moiety as defined herein; and,
wherein at least one of R^{3a}, R^{5a}, R^{6a} and Y^{a} comprises an in vivo imaging moiety as defined herein.

For preferred in vivo imaging agents of Formula Ia:
one of R^{3a}, R^{5a} or R^{6a} or Y^{a} comprises an in vivo imaging moiety chosen from ¹⁸F, ¹²³I or a chelating group comprising a chelated radioactive or paramagnetic metal ion; or,
one of R⁹ or R¹⁰ is an in vivo imaging moiety selected from C₁₋₆ [¹⁸F]fluoroalkyl or C₁₋₆ [¹¹C]alkyl; and,
   the remaining groups are as defined above for Formula Ia.

The structure and synthesis of in vivo imaging agents of Formula Ia are provided in WO 2007/064773. Also, Mathis et al (J Med Chem 2003; 46: 2740-54) and Klunk et al (Ann. Neurol. 2004; 55 306-19) describe synthesis of a particular ¹¹C-labelled compound of Formula Ia; and Serdons et al (2006 J. Nuc. Med.; 47(Supp1.1): 31P) reports direct aromatic nucleophilic substitution of a ¹⁸F-atom for a nitro group to form a ¹⁸F-labelled compound of Formula Ia. These reported methods can be easily adapted by the skilled person e.g. using known methods of labelling as described above, to obtain a range of in vivo imaging agents of Formula Ia.

In another preferred embodiment, said in vivo imaging agent is a compound of Formula Ib:

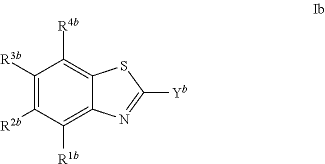

or a salt or solvate thereof, wherein:
each R^{1b}-R^{4b} is independently hydrogen, or an R group as defined above for R¹-R⁴, or R^{1b}-R^{4b} independently comprises an in vivo imaging moiety as defined herein;
Y^{b} is —R¹¹R¹², wherein R¹¹ is either a bond or a C₁₋₆ straight or branched alkenylene linker, and R¹² is a C₃₋₁₀ 5- to 10-membered aryl ring system having 0-3 heteroatoms selected from S, O and N, and 0-5 substituents each of which is an R group as defined above for R¹-R⁴, or Y^{b} comprises an in vivo imaging moiety as defined herein; and,
wherein at least one of R^{1b}-R^{4b} and Y^{b} comprises an in vivo imaging moiety as defined herein.

The term “alkenylene” refers to a divalent radical of a branched or unbranched unsaturated hydrocarbon group having from 2 to 6 carbon atoms, and having at least 1 and preferably from 1-6 sites of vinyl unsaturation. This term is exemplified by groups such as ethenylene (—CH═CH—), the propenylene isomers (e.g., —CH₂CH═CH— and —C(CH₃) CH—).

Preferably for Formula Ib:
R¹¹ is a C₁₋₆ straight or branched alkenylene linker;
R¹² is a C₃₋₁₀ 5- to 10-membered aryl ring system having 1 or 2 heteroatoms selected from S and N, and 0-5 substituents each of which is an R group as defined above, or R¹² comprises an in vivo imaging moiety as defined herein; and,
wherein one of R^{1b}-R^{4b}, or R¹² comprises an in vivo imaging moiety as defined herein.

Most preferably for Formula Ib:
R¹² is one of the following groups:

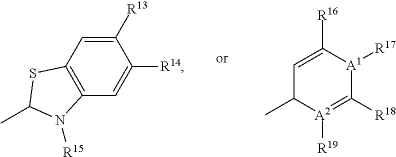

wherein:
A¹ is N or CH; A² is N or C; wherein at least one of A¹ or A² is N;
R¹³, R¹⁴, and R¹⁶⁻¹⁹ are independently selected from hydrogen, C₁₋₃ alkyl, or comprise an in vivo imaging moiety as defined herein; or R¹⁶ and R¹⁷, when A¹ is CH, together with A¹ and the carbon to which R¹⁶ is attached, form a benzene ring; or R¹⁸ and R¹⁹, when A² is C, together with A² and the carbon to which R¹⁸ is attached, form a benzene ring;
R¹⁵ is hydrogen or C₁₋₃ alkyl or comprises an in vivo imaging moiety as defined herein; and,

wherein at least one of R^{1b}-R^{4b}, or R¹³-R¹⁹ comprises an in vivo imaging moiety as defined herein.

Especially preferably for Formula Ib:
one of R^{1b}-R^{4b} is an in vivo imaging moiety chosen from ¹⁸F, ¹²³I or a chelating group comprising a chelated radioactive or paramagnetic metal ion; or,
one of R¹¹ or R¹² is an in vivo imaging moiety chosen from a chelating group comprising a chelated radioactive or paramagnetic metal ion, C₁₋₆ [¹⁸F]fluoroalkyl, or [¹¹C]methyl; and,
the remaining groups are as defined above.

Examples of preferred in vivo imaging moieties of Formula Ib are labelled versions of the compounds described by Volkova et al (Bioorg. Med. Chem. 2008; 16: 1452-9). To obtain labelled versions of these compounds, straightforward application of known methods of introducing in vivo imaging moieties can be used, as described earlier.

### Congo Red Derivatives

WO 02/074347 discloses ^{99m}Tc-labelled Congo Red derivatives suitable for use in in vivo imaging of amyloid plaques. Amyloid plaques are present in a range of diseases, most notably Alzheimer's disease. The present inventors propose that these and other Congo Red derivatives are also suitable for application in the method of the present invention.

Therefore, in an alternative preferred embodiment, said α-synuclein binder is a compound of Formula II:

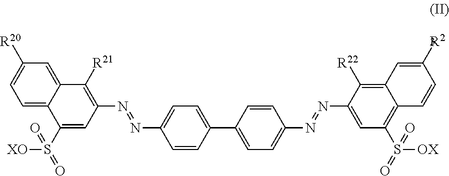

or a salt or solvate thereof, wherein:
R²⁰⁻²³ are independently selected from H, C₁₋₆ alkyl, halo, C₁₋₆ haloalkyl, amino, and C₁₋₆ aminoalkyl, or at least one of R²⁰⁻²³ comprises an in vivo imaging moiety as defined herein; and,
X represents a cation selected from hydrogen, potassium, and sodium.

Preferably, one of R²⁰-R²³ is an in vivo imaging moiety as defined above, and the remaining R²⁰-R²³ groups are as defined above.

Most preferably, one of R²⁰ or R²³ is ¹⁸F or ¹²³I; or; one of R²¹ or R²² is a chelating group comprising a chelated radioactive or paramagnetic metal ion, a C₁₋₆ [¹⁸F]-fluoroalkyl group, or [¹¹ C]methyl group.

Methods to obtain ^{99m}Tc labelled in vivo imaging agents of Formula II are described in WO 02/1074347, The methods therein can be easily adapted using the above-described techniques for adding metal-chelate complexes and other in vivo imaging moieties to obtain further in vivo imaging agents suitable for use in the present invention.

### Antibodies

In a further alternative preferred embodiment, said α-synuclein binder is an antibody that specifically binds to α-synuclein.

An “antibody” refers to a full-length (i.e., naturally occurring or formed by normal immunoglobulin gene fragment recombinatorial processes) immunoglobulin molecule (e.g. an IgG antibody) or an immunologically active (i.e., specifically binding) portion of an immunoglobulin molecule, such as an antibody fragment.

An “antibody fragment” is a portion of an antibody such as F(ab)₂, Fab, Fv, sFv, and the like. Regardless of structure, an antibody fragment binds with the same antigen that is recognized by the intact antibody. The term “antibody fragment” also includes any synthetic or genetically-engineered protein that acts like an antibody by binding to a specific antigen to form a complex. For example, antibody fragments include isolated fragments consisting of the variable regions, such as the Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region.

The phrase “specifically binds” refers to a binding reaction which is determinative of the presence of the protein in the presence of a heterogeneous population of proteins. Thus, under designated conditions, a specified ligand binds preferentially to a particular protein and does not bind in a significant amount to other proteins present in the sample. A molecule such as antibody that specifically binds to a protein often has an association constant of at least 10⁶ M⁻¹ or 10⁷ M⁻¹, preferably 10⁸ M⁻¹ to 10⁹ M⁻¹, and more preferably, about 10¹⁰ M⁻¹ to 10¹¹M⁻¹ or higher. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

There are numerous disclosures in the art of methods to obtain and characterise antibodies specific for α-synuclein suitable for use in the method of the present invention. The following paragraphs summarise a selection of these disclosures.

A number of studies have used antibodies that specifically bind to α-synuclein in the characterisation of LB in brain tissue samples taken from PD and DLB patients. Baba et al (1998 μm. J. Pathol.; 152: 879-84) characterised α-synuclein in LB using a monoclonal antibody raised against LB purified from DLB brains. Arima et al (1998 Brain Res; 808: 93-100) raised antibodies against the N-terminal, non-amyloid component (NAC) domain and C-terminal of α-synuclein. When characterised, the antibodies raised against the NAC domain and the C-terminal were found to be specific for α-synuclein over β-synuclein. In another study around the same time, Spillantini et al (1997 Nature; 388: 839-40) raised antibodies against either residues 11-34 or residues 116-131 of α-synuclein, both of which were found to specifically bind to α-synuclein and not to β-synuclein. Crowther et al (2000 Neurosci Lett; 292: 128-130) raised antibodies against the carboxy-terminal region of α-synuclein, which were found to label isolated filaments of α-synuclein along their entire lengths, whereas an antibody directed against the amino-terminal region of α-synuclein only labelled one filament end.

WO 99/50300 provides a monoclonal antibody raised against LB which is specific for α-synuclein. WO 99/50300 teaches that a suitably labelled version of this monoclonal antibody can be used in an in vitro assay to detect α-synuclein present in a biological sample. WO 2008/0175838 also relates to antibodies specific for α-synuclein, and discloses that the antibodies may be labelled with a fluorescent, radioactive or paramagnetic label for in vivo detection of LB in the brain of a subject. WO 2005/013889 provides methods of in vivo imaging LB in a patient by administration of an α-synuclein-specific antibody labelled with a paramagnetic or radioactive label. The antibodies of WO 2008/0175838 and WO 2005/013889 labelled with in vivo imaging moieties are suitable for use in the present invention.

In order to conjugate an antibody to an in vivo imaging moiety that is a radioactive metal or a paramagnetic ion, the antibody can be reacted with a reagent having a long tail to which is attached one or more chelating groups for binding the ions. Such a tail can be a polymer such as a polylysine, polysaccharide, or other derivatized or derivatizable chain having pendant groups to which can be bound one or more suitable chelating groups as described above. Chelates are coupled to the peptide antigens using standard chemistries. The chelate is normally linked to the antibody by: a group which enables formation of a bond to the molecule with minimal loss of immunoreactivity and minimal aggregation and/or internal cross-linking. Other, more unusual, methods and reagents for conjugating chelates to antibodies are disclosed in U.S. Pat. No. 4,824,659.

For the present invention, preferred in vivo imaging moieties for labelling α-synuclein-specific antibodies are ¹⁸F, ¹²³I and ^{99m}Tc.

An in vivo imaging moiety can be attached at the hinge region of a reduced antibody component via disulfide bond formation. As an alternative, such moieties can be attached to the antibody component using a heterobifunctional cross linker, such as N-succinyl 3-(2-pyridyldithio)proprionate (SPDP). General techniques for such conjugation are well-known in the art. See, for example, Wong, “Chemistry of Protein Conjugation and Cross-Linking” (CRC Press 1991). Alternatively, the in vivo imaging moiety can be conjugated via a carbohydrate moiety in the Fc region of the antibody.

Antibodies can be labelled with such reagents using protocols and techniques known and practiced in the art. See, for example, Wenzel and Meares, “Radioimmunoimaging and Radioimmunotherapy”, Elsevier, N.Y., 1983; Colcer et al 1986 Meth. Enzymol., 121: 802-816; and “Monoclonal Antibodies for Cancer Detection and Therapy”, Eds. Baldwin et al., Academic Press, 1985, pp. 303-316, for techniques relating to the radiolabeling of antibodies.

### Pharmaceutical Composition

The in vivo imaging agent of the invention is preferably administered as a “pharmaceutical composition” which comprises said in vivo imaging agent, together with a biocompatible carrier, in a form suitable for mammalian administration.

The “biocompatible carrier” is a fluid, especially a liquid, in which the in vivo imaging agent as defined herein is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can be withdrawn into clinical grade syringes at various time intervals during the viable lifetime of the preparation to suit the clinical situation. Pre-filled syringes are designed to contain a single human dose, or “unit dose”, and are therefore preferably a disposable or other syringe suitable for clinical use. Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition may be prepared from a kit. Alternatively, it may be prepared under aseptic manufacture conditions to give the desired sterile product. The pharmaceutical composition may also be prepared under non-sterile conditions, followed by terminal sterilisation using e.g. gamma-irradiation, autoclaving, dry heat or chemical treatment (e.g. with ethylene oxide).

### Diagnosis and Treatment Monitoring

The protein α-synuclein is found in healthy nerve cells as an unfolded membrane-bound protein. In response to pathological stimuli during the pathophysiology of a synucleinopathy, α-synuclein detaches from the membrane and takes on a β-sheet conformation, leading to aggregation and formation of LB and LN. A “synucleinopathy” is a neurodegenerative disease characterised by the presence of α-synuclein deposits in the neurons and the glia. Parkinson's disease (PD), dementia with Lewy bodies (DLB) and multiple system atrophy (MSA) are known examples of synucleinopathies. It has been postulated that α-synuclein deposits are present in the ANS in the early stages of PD (Braak et al J. Neural Transm. 2003; 110: 517-36), and as such the method of the present invention is useful in the early diagnosis of PD.

The present invention therefore also provides a method for the determination of the presence of, or susceptibility to, PD, said method as described above in relation to the in vivo imaging agent of the invention. Early diagnosis of PD, or of a susceptibility to PD, is advantageous as the disease process can be treated at early stage and treat disease before the onset of symptoms. Currently there is no such early diagnostic method such that by the time of diagnosis the patient has lost the majority of the nigrastriatal neurons controlling motor function, and application of neuroprotective agents is only beneficial for the remaining nigrastriatal neurons.

In a yet further aspect, the method of the present invention as described herein may be performed repeatedly, each performance being at a temporally distinct point in time, and wherein the images obtained in step (iv) are compared. Such a method is useful in monitoring the progression of PD. In a preferred embodiment, the method is performed before, during and/or after implementation of a treatment regimen, in order to determine the effectiveness of said treatment regimen.

In another aspect, the present invention provides an α-synuclein binder as defined herein for use in the preparation of an in vivo imaging agent for use in any of the methods defined herein.

In a further aspect, the present invention provides an in vivo imaging agent as defined herein for use in the manufacture of a medicament suitable for use in either the method of diagnosis, or the method of treatment monitoring as described above.

What is claimed is:

1) A method to diagnose the early stages of Parkinson's disease (PD), said method comprising:
(i) administering to a subject a detectable quantity of an in vivo imaging agent, wherein said in vivo imaging agent comprises an α-synuclein binder labelled with an in vivo imaging moiety, and wherein said in vivo imaging agent binds to α-synuclein with a binding affinity of 0.1 nM-50 μM;
(ii) allowing said administered in vivo imaging agent of step (i) to bind to α-synuclein deposits in the autonomic nervous system (ANS) of said subject;
(iii) detecting signals emitted by said bound in vivo imaging agent of step (ii) using an in vivo imaging method;
(iv) generating an image representative of the location and/or amount of said signals; and,
(v) using the image generated in step (iv) to determine of the presence of, or susceptibility to, PD.

2) The method as defined in claim 1 wherein said in vivo imaging moiety is:
(i) a radioactive metal ion;
(ii) a paramagnetic metal ion;
(iii) a gamma-emitting radioactive halogen;
(iv) a positron-emitting radioactive non-metal; or
(v) a reporter suitable for in vivo optical imaging.

3) The method as defined in claim 1 wherein said α-synuclein binder is a compound of Formula I or Formula I(i): 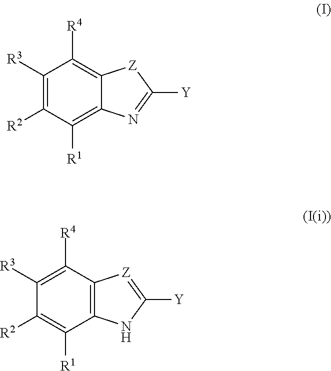
or a salt or solvate thereof, wherein:
R¹⁻⁴ are each independently hydrogen or an R group selected from C₁₋₆ alkyl, C₂₋₆ alkenyl, C₂₋₆ alkynyl, C₁₋₆ alkoxy, C₄₋₆ cycloalkyl, hydroxyl, C₁₋₆ hydroxyalkyl, C₂₋₆ hydroxyalkenyl, C₂₋₆ hydroxyalkynyl, thiol, C₁₋₆ thioalkyl, C₂₋₆ thioalkenyl, C₂₋₆ thioalkynyl, C₁₋₆ thioalkoxy, carboxyl, C₁₋₆ carboxyalkyl, halo, C₁₋₆ haloalkyl, C₂₋₆ haloalkenyl, C₂₋₆ haloalkynyl, C₁₋₆ haloalkoxy, amino, C₁₋₆ aminoalkyl, C₂₋₆ aminoalkenyl, C₂₋₆ aminoalkynyl, C₁₋₆-aminoalkoxy, cyano, C₁₋₆ cyanoalkyl, C₂₋₆ cyanoalkenyl, C₂₋₆ cyanoalkynyl, and C₁₋₆ cyanoalkoxy; nitro, C₁₋₆ nitroalkyl, C₂₋₆ nitroalkenyl, C₂₋₆ nitroalkynyl, C₁₋₆ nitroalkoxy, and —OCH₂OR′, wherein R′ is H or C₁₋₆ alkyl;
Y is a 5- to 10-membered aryl ring system optionally containing (i) 0-3 heteroatoms selected from S, O and N, and (ii) 0-5 substituents each of which is an R group as defined for R¹⁻⁴;
in Formula I, Z is S, O, or NR″ wherein R″ is hydrogen or C₁₋₃ alkyl; and,
in Formula I(i), Z is CR″ wherein R″ is hydrogen or C₁₋₃ alkyl

4) The method as defined in claim 1 wherein said in vivo imaging agent is a compound of Formula Ia: 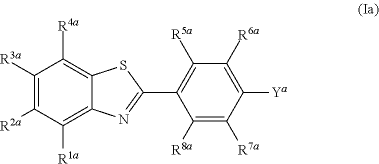
or a salt or solvate thereof, wherein:
each R^{1a}-R^{8a} is independently hydrogen or an R group selected from C₁₋₆ alkyl, C₂₋₆ alkenyl, C₂₋₆ alkynyl, C₁₋₆ alkoxy, C₄₋₆ cycloalkyl, hydroxyl, C₁₋₆ hydroxyalkyl, C₂₋₆ hydroxyalkenyl, C₂₋₆ hydroxyalkynyl, thiol, C₁₋₆ thioalkyl, C₂₋₆ thioalkenyl, C₂₋₆ thioalkynyl, C₁₋₆ thioalkoxy, carboxyl, C₁₋₆ carboxyalkyl, halo, C₁₋₆ haloalkyl, C₂₋₆ haloalkenyl, C₂₋₆ haloalkynyl, C₁₋₆ haloalkoxy, amino, C₁₋₆ aminoalkyl, C₂₋₆ aminoalkenyl, C₂₋₆ aminoalkynyl, C₁₋₆ aminoalkoxy, cyano, C₁₋₆ cyanoalkyl, C₂₋₆ cyanoalkenyl, C₂₋₆ cyanoalkynyl, and C₁₋₆ cyanoalkoxy; nitro, C₁₋₆ nitroalkyl, C₂₋₆ nitroalkenyl, C₂₋₆ nitroalkynyl, C₁₋₆ nitroalkoxy, and —OCH₂OR′, wherein R′ is H or C₁₋₆ alkyl, wherein said R group optionally comprises an in vivo imaging moiety; and,
Y^{a} is hydrogen, C₁₋₆ alkyl, halo, hydroxyl, C₁₋₆ hydroxyalkyl, thiol, C₁₋₆ thioalkyl, or an amino group —NR⁹R¹⁰, wherein R⁹ and R¹⁰ are independently hydrogen or an R group as defined for R^{1a}-R^{8a} above, in claim 3, or wherein said Y^{a} optionally comprises an in vivo imaging moiety; and
wherein at least one of R^{1a}-R^{8a} and Y^{a} comprises an in vivo imaging moiety.

5-7. (canceled)

8) The method as defined in claim 1 wherein said in vivo imaging agent is a compound of Formula Ib: 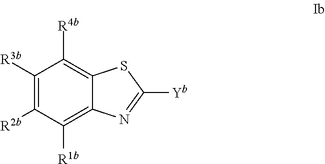
or a salt or solvate thereof, wherein:
each R^{1b}-R^{4b} is independently hydrogen, or an R group selected from C₁₋₆ alkyl, C₂₋₆ alkenyl, C₂₋₆ alkynyl, C₁₋₆ alkoxy, C₄₋₆ cycloalkyl, hydroxyl, C₁₋₆ hydroxyalkyl, C₂₋₆ hydroxyalkenyl, C₂₋₆ hydroxyalkynyl, thiol, C₁₋₆ thioalkyl, C₂₋₆ thioalkenyl, C₂₋₆ thioalkynyl, C₁₋₆ thioalkoxy, carboxyl, C₁₋₆ carboxyalkyl, halo, C₁₋₆ haloalkyl, C₂₋₆ haloalkenyl, C₂₋₆ haloalkynyl, C₁₋₆ haloalkoxy, amino, C₁₋₆ aminoalkyl, C₂₋₆ aminoalkenyl, C₂₋₆ aminoalkynyl, C₁₋₆ aminoalkoxy, cyano, C₁₋₆ cyanoalkyl, C₂₋₆ cyanoalkenyl, C₂₋₆ cyanoalkynyl, and C₁₋₆ cyanoalkoxy; nitro, C₁₋₆ nitroalkyl, C₂₋₆ nitroalkenyl, C₂₋₆ nitroalkynyl, C₁₋₆ nitroalkoxy, and —OCH₂OR′, wherein R′ is H or C₁₋₆ alkyl, wherein said R group optionally comprises an in vivo imaging moiety;
Y^{b} is —R¹¹R¹², wherein R¹¹ is either a bond or a C₁₋₆ straight or branched alkenylene linker, and R¹² is a 5- to 10-membered aryl ring system optionally containing (i) 0-3 heteroatoms selected from S, O and N, and (ii) 0-5 substituents each of which is an R group as defined above for R^{1b}-R^{4b}, wherein Y^{b} optionally comprises an in vivo imaging moiety; and,
wherein at least one of R^{1b}-R^{4b} Y^{b} comprises an in vivo imaging moiety.

9-11. (canceled)

12) The method as defined in claim 1 which wherein said in vivo imaging agent is a compound of Formula II: 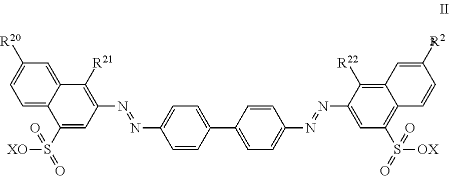
or a salt or solvate thereof, wherein:
R²⁰⁻²³ are independently selected from H, C₁₋₆ alkyl, halo, C₁₋₆ haloalkyl, amino, and C₁₋₆ aminoalkyl, wherein at least one of R²⁰⁻²³ comprises an in vivo imaging moiety; and,
X represents hydrogen, potassium cation, or sodium cation.

13-15. (canceled)

16) The method as defined in claim 1 wherein said α-synuclein binder is an antibody that specifically binds to α-synuclein.

17. (canceled)

18) The method as defined in claim 1 wherein in step (ii), said α-synuclein deposits are present in the enteric nervous system.

19) The method as defined in claim 1 wherein in step (ii), said α-synuclein deposits are Lewy bodies (LB) and/or Lewy neurites (LN).

20) The method as defined in claim 1 wherein said subject of step (i) of said method is a mammal.

21) The method as described in claim 1, wherein said in vivo imaging agent is administered in step (i) as a pharmaceutical composition, said pharmaceutical composition comprising said in vivo imaging agent and a biocompatible carrier suitable for mammalian administration.

22. (canceled)

23) A method for monitoring the progression of Parkinson's disease comprising the method as defined in claim 1 performed repeatedly, each performance being at a temporally distinct point in time, wherein the images obtained in step (iv) are compared to determine progression of PD.

24) The method as defined in claim 23 wherein the method is performed before, during and/or after implementation of a treatment regimen.

25-26. (canceled)
